(19) United States
(12) Patent Application Publication
(10) Pub. No.: US 2011/0124865 A1
(43) Pub. Date: May 26, 2011

(54) PROCESS FOR THE PREPARATION OF SUBSTITUTED PHENYLALANINES

(76) Inventors: Shinya IIMURA, Tsukuba (JP); Wenxue WU, Princeton Junction, NJ (US); Matthew Mangzhu ZHAO, Edison, NJ (US)

(21) Appl. No.: 12/948,901

(22) Filed: Nov. 18, 2010

Related U.S. Application Data
(60) Provisional application No. 61/262,834, filed on Nov. 19, 2009.

(51) Int. Cl. C07D 239/47; C07C 227/16

(52) U.S. Cl. 544/321; 562/443

(57) ABSTRACT

Intermediates and synthetic processes for the preparation of substituted phenylalanine-based compounds are disclosed.

This application claims priority to U.S. provisional patent application No. 61/262,834, filed Nov. 19, 2010, the entirety of which is incorporated herein by reference.

### 1. FIELD OF THE INVENTION

This invention relates to synthetic processes used to make substituted phenylalanine-based compounds.

### 2. BACKGROUND

The enzyme tryptophan hydroxylase (TPH) catalyzes the rate limiting step of the biosynthesis of serotonin. Inhibitors of the enzyme have been proposed as potential treatments of a variety of diseases and disorders, including irritable bowel syndrome and carcinoid syndrome. See, e.g., U.S. patent application publication no. US-2007-0191370-A1; U.S. Pat. No. 7,553,840. Although large scale methods of preparing these compounds have been disclosed (see, e.g., U.S. patent application publication no. US-2009-0048280-A1), additional methods are desired.

### 3. SUMMARY OF THE INVENTION

This invention encompasses methods of preparing compounds of formula 1:

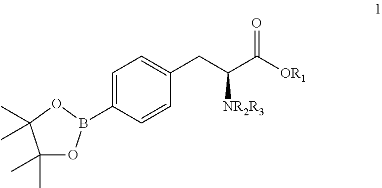

wherein: R₁ is hydrogen or optionally substituted alkyl, alkyl-aryl, or aryl; R₂ is hydrogen or a protecting group; and R₃ is a protecting group. Compounds of formula 1 can be used to preparing compounds of formula 2:

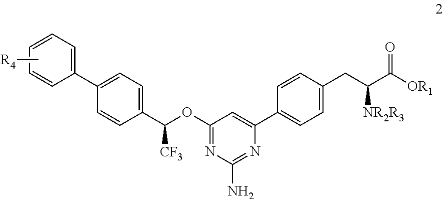

and pharmaceutically acceptable salts thereof, wherein R₄ is halo or optionally substituted alkyl, aryl, or alkoxy. Other methods useful in preparing compounds of formula 2 are also encompassed by the invention.

### 4. DETAILED DESCRIPTION

This invention is directed, in part, to improved methods of synthesizing the TPH inhibitor (S)-2-amino-3-(4-(2-amino-6-((R)-2,2,2-trifluoro-1-(3′-methoxybiphenyl-4-yl)ethoxy)pyrimidin-4-yl)phenyl)propanoic acid and intermediates useful in its synthesis. See U.S. patent application publication no. US-2009-0048280-A1, the entirety of which is incorporated herein by reference.

### 4.1. DEFINITIONS

Unless otherwise indicated, the term “alkenyl” means a straight chain, branched and/or cyclic hydrocarbon having from 2 to 20 (e.g., 2 to 10 or 2 to 6) carbon atoms, and including at least one carbon-carbon double bond. Representative alkenyl moieties include vinyl, allyl, 1-butenyl, 2-butenyl, isobutylenyl, 1-pentenyl, 2-pentenyl, 3-methyl-1-butenyl, 2-methyl-2-butenyl, 2,3-dimethyl-2-butenyl, 1-hexenyl, 2-hexenyl, 3-hexenyl, 1-heptenyl, 2-heptenyl, 3-heptenyl, 1-octenyl, 2-octenyl, 3-octenyl, 1-nonenyl, 2-nonenyl, 3-nonenyl, 1-decenyl, 2-decenyl and 3-decenyl.

Unless otherwise indicated, the term “alkyl” means a straight chain, branched and/or cyclic (“cycloalkyl”) hydrocarbon having from 1 to 20 (e.g., 1 to 10 or 1 to 4) carbon atoms. Alkyl moieties having from 1 to 4 carbons are referred to as “lower alkyl.” Examples of alkyl groups include, but are not limited to, methyl, ethyl, propyl, isopropyl, n-butyl, t-butyl, isobutyl, pentyl, hexyl, isohexyl, heptyl, 4,4-dimethylpentyl, octyl, 2,2,4-trimethylpentyl, nonyl, decyl, undecyl and dodecyl. Cycloalkyl moieties may be monocyclic or multicyclic, and examples include cyclopropyl, cyclobutyl, cyclopentyl, cyclohexyl, and adamantyl. Additional examples of alkyl moieties have linear, branched and/or cyclic portions (e.g., 1-ethyl-4-methyl-cyclohexyl). The term “alkyl” includes saturated hydrocarbons as well as alkenyl and alkynyl moieties.

Unless otherwise indicated, the term “alkylaryl” or “alkyl-aryl” means an alkyl moiety bound to an aryl moiety.

Unless otherwise indicated, the term “alkylheteroaryl” or “alkyl-heteroaryl” means an alkyl moiety bound to a heteroaryl moiety.

Unless otherwise indicated, the term “alkylheterocycle” or “alkyl-heterocycle” means an alkyl moiety bound to a heterocycle moiety.

Unless otherwise indicated, the term “alkynyl” means a straight chain, branched or cyclic hydrocarbon having from 2 to 20 (e.g., 2 to 20 or 2 to 6) carbon atoms, and including at least one carbon-carbon triple bond. Representative alkynyl moieties include acetylenyl, propynyl, 1-butynyl, 2-butynyl, 1-pentynyl, 2-pentynyl, 3-methyl-1-butynyl, 4-pentynyl, 1-hexynyl, 2-hexynyl, 5-hexynyl, 1-heptynyl, 2-heptynyl, 6-heptynyl, 1-octynyl, 2-octynyl, 7-octynyl, 1-nonynyl, 2-nonynyl, 8-nonynyl, 1-decynyl, 2-decynyl and 9-decynyl.

Unless otherwise indicated, the term “alkoxy” means an —O-alkyl group. Examples of alkoxy groups include, but are not limited to, —OCH₃, —OCH₂CH₃, —O(CH₂)₂CH₃, —O(CH₂)₃CH₃, —O(CH₂)₄CH₃, and —O(CH₂)₅CH₃.

Unless otherwise indicated, the term “aryl” means an aromatic ring or an aromatic or partially aromatic ring system composed of carbon and hydrogen atoms. An aryl moiety may comprise multiple rings bound or fused together. Examples of aryl moieties include, but are not limited to, anthracenyl, azulenyl, biphenyl, fluorenyl, indan, indenyl, naphthyl, phenanthrenyl, phenyl, 1,2,3,4-tetrahydro-naphthalene, and tolyl.

Unless otherwise indicated, the term “arylalkyl” or “aryl-alkyl” means an aryl moiety bound to an alkyl moiety.

Unless otherwise indicated, the terms “halogen” and “halo” encompass fluorine, chlorine, bromine, and iodine.

Unless otherwise indicated, the term “heteroalkyl” refers to an alkyl moiety in which at least one of its carbon atoms has been replaced with a heteroatom (e.g., N, O or S).

Unless otherwise indicated, the term “heteroaryl” means an aryl moiety wherein at least one of its carbon atoms has been replaced with a heteroatom (e.g., N, O or S). Examples include, but are not limited to, acridinyl, benzimidazolyl, benzofuranyl, benzoisothiazolyl, benzoisoxazolyl, benzoquinazolinyl, benzothiazolyl, benzoxazolyl, furyl, imidazolyl, indolyl, isothiazolyl, isoxazolyl, oxadiazolyl, oxazolyl, phthalazinyl, pyrazinyl, pyrazolyl, pyridazinyl, pyridyl, pyrimidinyl, pyrimidyl, pyrrolyl, quinazolinyl, quinolinyl, tetrazolyl, thiazolyl, and triazinyl.

Unless otherwise indicated, the term “heteroarylalkyl” or “heteroaryl-alkyl” means a heteroaryl moiety bound to an alkyl moiety.

Unless otherwise indicated, the term “heterocycle” refers to an aromatic, partially aromatic or non-aromatic monocyclic or polycyclic ring or ring system comprised of carbon, hydrogen and at least one heteroatom (e.g., N, O or S). A heterocycle may comprise multiple (i.e., two or more) rings fused or bound together. Heterocycles include heteroaryls. Examples include, but are not limited to, benzo[1,3]dioxolyl, 2,3-dihydro-benzo[1,4]dioxinyl, cinnolinyl, furanyl, hydantoinyl, morpholinyl, oxetanyl, oxiranyl, piperazinyl, piperidinyl, pyrrolidinonyl, pyrrolidinyl, tetrahydrofuranyl, tetrahydropyranyl, tetrahydropyridinyl, tetrahydropyrimidinyl, tetrahydrothiophenyl, tetrahydrothiopyranyl and valerolactamyl.

Unless otherwise indicated, the term “heterocyclealkyl” or “heterocycle-alkyl” refers to a heterocycle moiety bound to an alkyl moiety.

Unless otherwise indicated, the term “heterocycloalkyl” refers to a non-aromatic heterocycle.

Unless otherwise indicated, the term “heterocycloalkylalkyl” or “heterocycloalkyl-alkyl” refers to a heterocycloalkyl moiety bound to an alkyl moiety.

Unless otherwise indicated, the term “pharmaceutically acceptable salts” refers to salts prepared from pharmaceutically acceptable non-toxic acids or bases including inorganic acids and bases and organic acids and bases. Suitable pharmaceutically acceptable base addition salts include, but are not limited to, metallic salts made from aluminum, calcium, lithium, magnesium, potassium, sodium and zinc or organic salts made from lysine, N,N′-dibenzylethylenediamine, chloroprocaine, choline, diethanolamine, ethylenediamine, meglumine (N-methylglucamine) and procaine. Suitable non-toxic acids include, but are not limited to, inorganic and organic acids such as acetic, alginic, anthranilic, benzenesulfonic, benzoic, camphorsulfonic, citric, ethenesulfonic, formic, fumaric, furoic, galacturonic, gluconic, glucuronic, glutamic, glycolic, hydrobromic, hydrochloric, isethionic, lactic, maleic, malic, mandelic, methanesulfonic, mucic, nitric, pamoic, pantothenic, phenylacetic, phosphoric, propionic, salicylic, stearic, succinic, sulfanilic, sulfuric, tartaric acid, and p-toluenesulfonic acid. Specific non-toxic acids include hydrochloric, hydrobromic, phosphoric, sulfuric, and methanesulfonic acids. Examples of specific salts thus include hydrochloride and mesylate salts. Others are well known in the art. See, e.g., Remington's Pharmaceutical Sciences (18th ed., Mack Publishing, Easton Pa.: 1990) and Remington: The Science and Practice of Pharmacy (19th ed., Mack Publishing, Easton Pa.: 1995).

Unless otherwise indicated, the term “protecting group” or “protective group,” when used to refer to part of a molecule subjected to a chemical reaction, means a chemical moiety that is not reactive under the conditions of that chemical reaction, and which may be removed to provide a moiety that is reactive under those conditions. Protecting groups are well known in the art. See, e.g., Greene, T. W. and Wuts, P. G. M., Protective Groups in Organic Synthesis (3^{rd} ed., John Wiley & Sons: 1999); Larock, R. C., Comprehensive Organic Transformations (2^{nd} ed., John Wiley & Sons: 1999).

Unless otherwise indicated, the term “substituted,” when used to describe a chemical structure or moiety, refers to a derivative of that structure or moiety wherein one or more of its hydrogen atoms is substituted with a chemical moiety or functional group such as, but not limited to, alcohol, aldehylde, alkoxy, alkanoyloxy, alkoxycarbonyl, alkenyl, alkyl (e.g., methyl, ethyl, propyl, t-butyl), alkynyl, alkylcarbonyloxy (—OC(O)alkyl), amide (—C(O)NH-alkyl- or -alkylNHC(O)alkyl), amidinyl (—C(NH)NH-alkyl or —C(NR)NH₂), amine (primary, secondary and tertiary such as alkylamino, arylamino, arylalkylamino), aroyl, aryl, aryloxy, azo, carbamoyl (—NHC(O)O-alkyl- or —OC(O)NH-alkyl), carbamyl (e.g., CONH₂, as well as CONH-alkyl, CONH-aryl, and CONH-arylalkyl), carbonyl, carboxyl, carboxylic acid, carboxylic acid anhydride, carboxylic acid chloride, cyano, ester, epoxide, ether (e.g., methoxy, ethoxy), guanidino, halo, haloalkyl (e.g., —CCl₃, —CF₃, —C(CF₃)₃), heteroalkyl, hemiacetal, imine (primary and secondary), isocyanate, isothiocyanate, ketone, nitrile, nitro, oxo, phosphodiester, sulfide, sulfonamido (e.g., SO₂NH₂), sulfone, sulfonyl (including alkylsulfonyl, arylsulfonyl and arylalkylsulfonyl), sulfoxide, thiol (e.g., sulfhydryl, thioether) and urea (—NHCONH-alkyl-).

Unless otherwise indicated, the term “include” has the same meaning as “include, but are not limited to,” and the term “includes” has the same meaning as “includes, but is not limited to.” Similarly, the term “such as” has the same meaning as the term “such as, but not limited to.”

Unless otherwise indicated, one or more adjectives immediately preceding a series of nouns is to be construed as applying to each of the nouns. For example, the phrase “optionally substituted alky, aryl, or heteroaryl” has the same meaning as “optionally substituted alky, optionally substituted aryl, or optionally substituted heteroaryl.”

It should be noted that a chemical moiety that forms part of a larger compound may be described herein using a name commonly accorded it when it exists as a single molecule or a name commonly accorded its radical. For example, the terms “pyridine” and “pyridyl” are accorded the same meaning when used to describe a moiety attached to other chemical moieties. Thus, the two phrases “XOH, wherein X is pyridyl” and “XOH, wherein X is pyridine” are accorded the same meaning, and encompass the compounds pyridin-2-ol, pyridin-3-ol and pyridin-4-ol.

It should also be noted that if the stereochemistry of a structure or a portion of a structure is not indicated with, for example, bold or dashed lines, the structure or the portion of the structure is to be interpreted as encompassing all stereoisomers of it. Similarly, names of compounds having one or more chiral centers that do not specify the stereochemistry of those centers encompass pure stereoisomers and mixtures thereof. Moreover, any atom shown in a drawing with unsatisfied valences is assumed to be attached to enough hydrogen atoms to satisfy the valences. In addition, chemical bonds depicted with one solid line parallel to one dashed line encompass both single and double (e.g., aromatic) bonds, if valences permit. This invention encompasses tautomers and solvates (e.g., hydrates) of the compounds disclosed herein.

### 4.2. METHODS OF SYNTHESIS

Methods of this invention are applicable to the preparation of (S)-2-amino-3-(4-(2-amino-6-((R)-2,2,2-trifluoro-1-(3′-methoxybiphenyl-4-yl)ethoxy)pyrimidin-4-yl)phenyl)propanoic acid and derivatives (e.g., protected precursors) and salts thereof. One method of preparing this specific compound is represented below in Scheme 1:

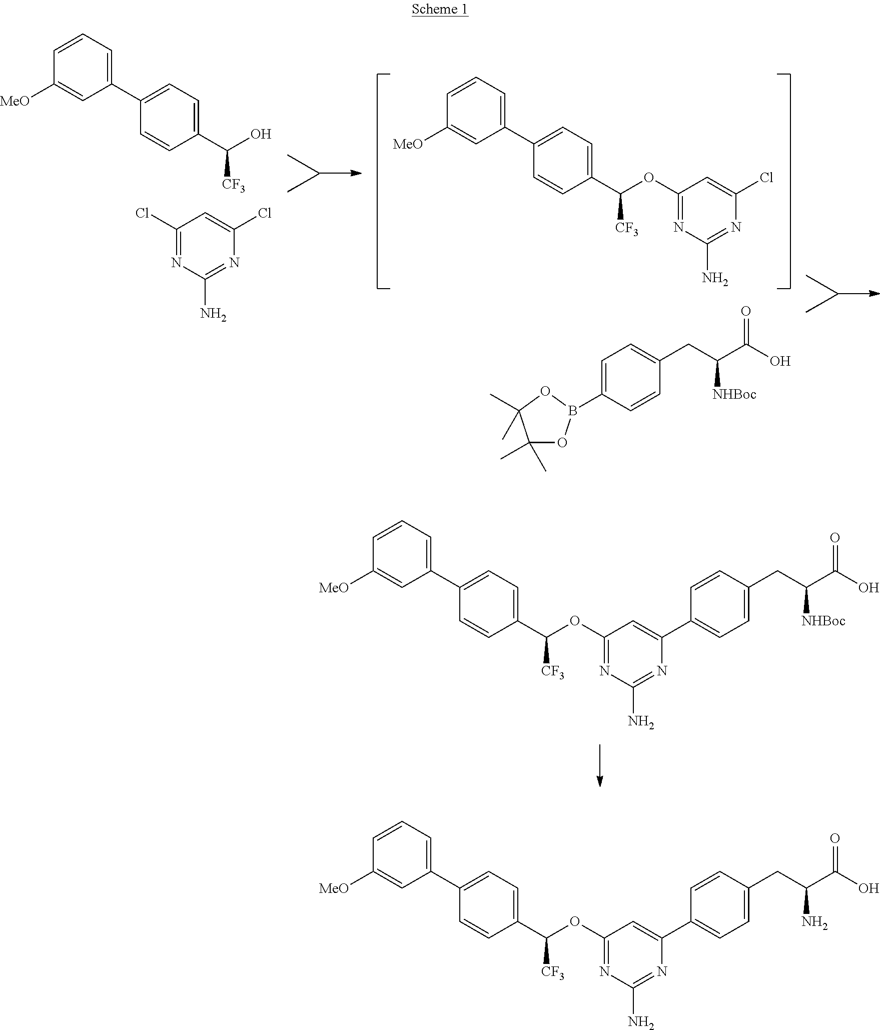

A particular embodiment of the invention encompasses the preparation of compounds of the formula:

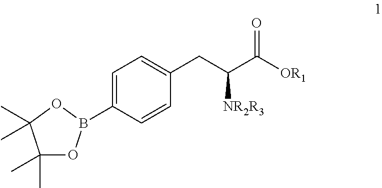

wherein R₁ is hydrogen or optionally substituted alkyl, alkyl-aryl, or aryl; R₂ is hydrogen or a protecting group; and R₃ is a protecting group, which comprises contacting a compound of the formula:

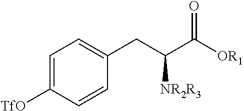

with pinacolborane. In a specific embodiment, R₁ is lower alkyl (e.g., methyl). Protecting groups for the amine moiety are known in the art, and include t-butoxycarbonyl (BOC), carbobenzyloxy (CBZ), acetyl, benzoyl, pivaloyl, benzyl, and alkyl. In particular embodiments, R₂ is hydrogen and R₃ is BOC. The reaction may be catalyzed by a palladium catalyst (e.g., PdCl₂(dppf).CH₂Cl₂, PdCl₂(dppf), and Pd(OAc)₂/dppf) in the presence of a tertiary amine (e.g., triethylamine, N-methylmorpholine (NMM), or diisopropylethylamine) in a suitable solvent. Suitable solvents include polar aprotic and non-polar solvents, such as dioxane, acetonitrile, toluene, 2-methyltetrahydrofuran, and mixtures thereof. In one embodiment, pinacolborane is produced in situ from a borane complex (e.g., borane-THF, borane-dimethyl sulfide, or a borane-amine such as borane-diethylaniline) and pinacol.

In a particular embodiment, the compound of formula 1 is contacted with a compound of formula 3:

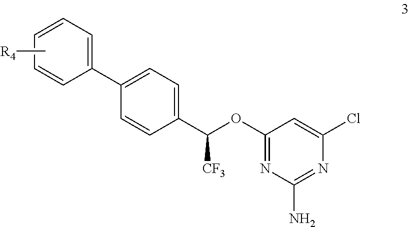

under conditions sufficient to afford a compound of formula 2:

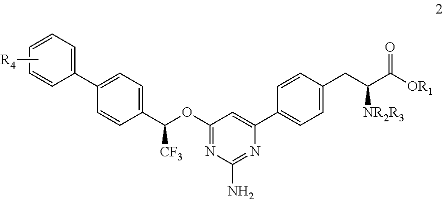

wherein R₄ is halo or optionally substituted alkyl, aryl, or alkoxy. In a particular embodiment, R₄ is methoxy. The reaction of compounds 1 and 3 may be catalyzed by a palladium catalyst (e.g., Pd(PPh₃)₂Cl₂/PPh₃, Pd(PPh₃)₂Cl₂, or Pd(dppf)Cl₂) in the presence of a base. Suitable bases include alkaline metal and alkaline earth metal carbonates, bicarbonates and phosphates, such as potassium carbonate, potassium bicarbonate, sodium carbonate, or sodium bicarbonate.

Compounds of formula 3 can be prepared by contacting a compound of the formula:

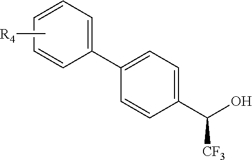

with 4,6-dichloropyrimidin-2-amine in the presence of a suitable base (e.g., cesium carbonate, potassium carbonate, or potassium phosphate). Suitable solvents include dioxane, t-butanol, t-amyl alcohol, DMF, DMAc, DMSO, NMP, and mixtures thereof.

### 5. EXAMPLES

While aspects of the invention can be understood from the following examples, they are not meant to limit its scope.

### 5.1. Preparation of (S)-2-(tert-butoxycarbonylamino)-3-(4-(4,4,5,5-tetramethyl-1,3,2-dioxaborolan-2-yl)phenyl)propanoic acid

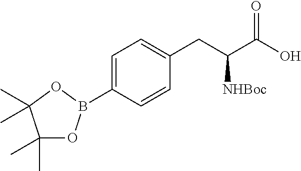

To a 1-L three-neck round bottom flask with a temperature controller, a mechanical stirrer, and N₂ inlet was charged pinacol (55.5 g, 470 mmol, 2.0 equiv) and dioxane (600 mL, 6×) at room temperature and stirred for 15 minutes to give a homogeneous solution. The solution was cooled to 5-10° C. and BH₃-PhNEt₂ (83.5 mL, 469 mmol, 2.0 equiv) was added over 15 min at 5-10° C. After stirred for 15 min at 10° C., it was warmed to room temperature and stirred for 4 hours at the same temperature to prepare a pinacolborane solution.

To a 3-L three-neck flask with a temperature controller, a mechanical stirrer, and a condenser protected under N₂ was charged the thick solution of (S)-methyl 2-(tert-butoxycarbonylamino)-3-(4-(trifluoromethylsulfonyloxy)phenyl)propanoate (149 g, 67.1 wt %: 100 g, 234 mmol), dioxane (300 mL, 3×), and N-methylmorpholine (NMM, 38.6 mL, 351 mmol, 1.5 equiv) at room temperature. After the mixture was degassed by conducting three vacuum/nitrogen purge cycles, PdCl₂(dppf).CH₂Cl₂ (955 mg, 1.17 mmol, 0.5 mol %) was added. Then the above pinacolborane solution was degassed by conducting three vacuum/nitrogen purge cycles and added to this reaction mixture at room temperature. The mixture was heated at 75° C. and stirred for 18 hours at the same temperature. The mixture was then cooled to room temperature (assay: 91.8 g (S)-methyl 2-(tert-butoxycarbonylamino)-3-(4-(4,4,5,5-tetramethyl-1,3,2-dioxaborolan-2-yl)phenyl)propanoate, 96.8% yield) and concentrated to ca. 3× under reduced pressure below 45° C. to give an oil. This thick solution was diluted with MTBE (500 mL, 5×) and washed with water (200 mL, 2×). The organic layer was then cooled to 0-5° C. and an aqueous solution of LiOH.H₂O (29.5 g, 703 mmol, 3.0 equiv) in water (800 mL, 8×) was added over 15 min at 0-10° C. and stirred for 20 min at 0-10° C. After separated the layers, the organic layer was extracted with water (200 mL, 2×). To the combined aqueous layer was added MTBE (500 mL, 5×) and cooled to 0-5° C. 6N HCl (^{˜}120 mL was used) was added dropwise to adjust the solution pH to 3 at 0-10° C. The layers was separated and the organic layer was washed with brine (200 mL, 2.0×). The resulting organic layer (partially emulsion) was dried over Na₂SO₄ (50 g, 0.5×), and concentrated to 2× under reduced pressure below 45° C. after pinacol (1.38 g, 11.7 mmol, 0.05 equiv) was added. The resulting thick solution was then heated at 45° C. and heptane (1 L, 10×) was slowly added to this solution. After the resulting slurry was stirred for 2 h at 45° C., slowly cooled to room temperature and stirred for 16 h at the same temperature. The solids were filtered and the wet cake was washed with heptane (100 mL, 1×, ×2). (S)-2-(tert-butoxycarbonylamino)-3-(4-(4,4,5,5-tetramethyl-1,3,2-dioxaborolan-2-yl)phenyl)propanoic acid was obtain as a white solid (75.4 g, 98 wt %, 81% yield from (S)-methyl 2-(tert-butoxycarbonylamino)-3-(4-(trifluoromethylsulfonyloxy)phenyl)propanoate, HPLC purity: 98.5%, chiral purity: >99.5%, KF: 0.31, Pd: 64 ppm) after dried under vacuum at 45° C.

### 5.2. Preparation of (S)-3-(4-(2-amino-6-((R)-2,2,2-trifluoro-1-(3′-methoxybiphenyl-4-yl)ethoxy)pyrimidin-4-yl)phenyl)-2-(tert-butoxycarbonylamino)propanoic acid

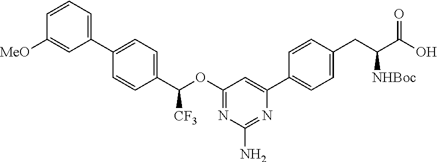

A suspension of alcohol ((R)-2,2,2-trifluoro-1-(3′-methoxybiphenyl-4-yl)ethanol, 30 g, 0.106 mol), pyrimidine (4,6-dichloropyrimidin-2-amine, 34.8 g, 0.212 mol) and cesium carbonate (34.6 g, 0.106 mol) in 1,4-dioxane (300 ml, 10×) was heated to 100° C. with good stirring. After stirred for 4 hours at 100° C., cesium carbonate (17.3 g, 0.053 mol) was added and further stirred for 14 h at 100° C. Cooled to 50° C., water (90 mL, 3×) was added and stirred for 30 min at room temperature. The organic layer was concentrated to a 5× solution and solid was removed by polish filtration. After diluted with toluene (300 mL, 10×) and concentrated to a 5× solution and heptane (150 mL, 5×) was added. After stirred for 2 hours at room temperature, removed solid by filtration. 1,4-dioxane was added and concentrated to prepare a solution of monochloride ((R)-4-chloro-6-(2,2,2-trifluoro-1-(3′-methoxybiphenyl-4-yl)ethoxy)pyrimidin-2-amine) in 1,4-dioxane.

To a 15× solution of monochloride ((R)-4-chloro-6-(2,2,2-trifluoro-1-(3′-methoxybiphenyl-4-yl)ethoxy)pyrimidin-2-amine, assuming 0.106 mol) in 1,4-dioxane was added boronic ester ((S)-2-(tert-butoxycarbonylamino)-3-(4-(4,4,5,5-tetramethyl-1,3,2-dioxaborolan-2-yl)phenyl)propanoic acid, 62.25 g, 0.159 mol), potassium bicarbonate (37.2 g, 0.372 mol) and water (90 mL, 3×) at room temperature. After degassed well (vacuum and nitrogen fill 3 times), PdCl₂(PPh₃)₂ (372 mg, 0.529 mmol) and triphenylphosphine (72 mg, 0.275 mmol) were added. The reaction mixture was then stirred for 8 h at 90° C. Cooled to room temperature, 2 N HCl was added slowly to adjust the pH to 3-4. After stirred for 30 min at room temperature, the organic layer was treated with activated carbon at 50° C. for 2 hours. After filtered though a pad of celite, the solution was then concentrated to a 3× under vacuum (50 mbar, 40° C.). CH₃CN (20×) was added and concentrated to ca. 10× suspension under the conditions (100 mbar, 40° C.). The slurry was filtered, washed with CH₃CN (10×), dried under vacuum at 40° C. to obtain the desired Boc acid as a white solid ((S)-3-(4-(2-amino-6-((R)-2,2,2-trifluoro-1-(3′-methoxybiphenyl-4-yl)ethoxy)pyrimidin-4-yl)phenyl)-2-(tert-butoxycarbonylamino)propanoic acid, 20.1 g, 98 wt %, 90% yield over 2 steps, HPLC purity: 97%, Pd: 69 ppm).

All of the publications (e.g., patents and patent applications) disclosed above are incorporated herein by reference in their entireties.

What is claimed is:

1. A method of preparing a compound of formula 1: 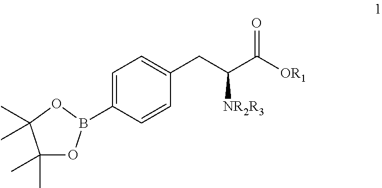
which comprises contacting a compound of the formula: 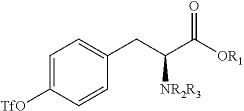
with pinacolborane under conditions sufficient to provide a compound of formula 1, wherein:
R₁ is hydrogen or optionally substituted alkyl, alkyl-aryl, or aryl;
R₂ is hydrogen or a protecting group; and
R₃ is a protecting group.

2. The method of claim 1, wherein R₁ is methyl.

3. The method of claim 1, wherein R₂ is hydrogen.

4. The method of claim 1, wherein R₃ is t-butoxycarbonyl (BOC), carbobenzyloxy (CBZ), acetyl, benzoyl, pivaloyl, benzyl, or alkyl.

5. The method of claim 1, wherein the conditions sufficient to provide a compound of formula 1 comprise the presence of a palladium catalyst.

6. The method of claim 5, wherein the palladium catalyst is PdCl₂(dppf).CH₂Cl₂, PdCl₂(dppf), or Pd(OAc)₂/dppf.

7. The method of claim 5, wherein the conditions sufficient to provide a compound of formula 1 comprise the presence of a tertiary amine (e.g., triethylamine, N-methylmorpholine (NMM), or diisopropylethylamine).

8. The method of claim 5, wherein the conditions sufficient to provide a compound of formula 1 comprise the presence of a polar aprotic or non-polar solvent.

9. The method of claim 8, wherein the solvent is dioxane, acetonitrile, toluene, or 2-methyltetrahydrofuran.

10. The method of claim 5, wherein pinacolborane is prepared in situ from a borane complex.

11. A method of preparing a compound of formula 2: 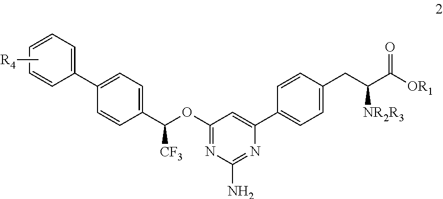
which comprises contacting a compound of formula 3: 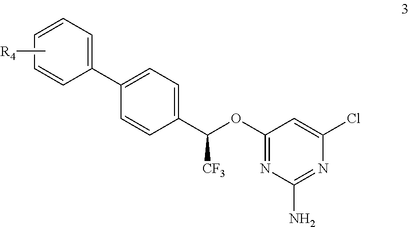
with a compound of the formula: 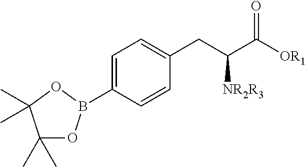
under conditions sufficient to provide a compound of formula 2, wherein:
R₁ is hydrogen or optionally substituted alkyl, alkyl-aryl, or aryl;
R₂ is hydrogen or a protecting group;
R₃ is a protecting group; and
R₄ is halo or optionally substituted alkyl, aryl, or alkoxy.

12. The method of claim 11, wherein R₁ is methyl.

13. The method of claim 11, wherein R₂ is hydrogen.

14. The method of claim 11, wherein R₃ is a protecting group that is t-butoxycarbonyl (BOC), carbobenzyloxy (CBZ), acetyl, benzoyl, pivaloyl, benzyl, or alkyl.

15. The method of claim 11, wherein R₄ is methoxy.

16. The method of claim 11, wherein the conditions sufficient to provide a compound of formula 2 comprise the presence of a palladium catalyst.

17. The method of claim 16, wherein the palladium catalyst is PdCl₂/PPh₃, Pd(OAc)₂/PPh₃, Pd(PPh₃)₂Cl₂, or Pd(dppf)Cl₂.

18. The method of claim 11, wherein the conditions sufficient to provide a compound of formula 2 comprise the presence of a base.

19. The method of claim 18, wherein the base is an alkaline metal or alkaline earth metal carbonate, bicarbonate or phosphate.

20. The method of claim 19, wherein the base is potassium carbonate, potassium bicarbonate, sodium carbonate, or sodium bicarbonate.

21. The method of claim 11, wherein the conditions sufficient to provide a compound of formula 2 comprise the presence of a polar solvent.

22. The method of claim 21, wherein the polar solvent is water, dioxane, isobutanol, t-amyl alcohol, DMF, DMAc, NMP, DMSO, or MIBK.

23. The method of 21 wherein the polar solvent comprises water.

24. The method of claim 11, wherein the compound of formula 3 is prepared by contacting a compound of the formula: 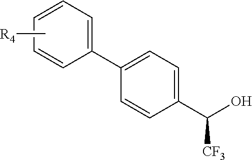
with 4,6-dichloropyrimidin-2-amine under conditions sufficient to provide a compound of formula 3.

25. The method of claim 24, wherein the conditions sufficient to provide a compound of formula 3 comprise the presence of a solvent.

26. The method of claim 25, wherein the solvent is dioxane, t-butanol, t-amyl alcohol, DMF, DMAc, DMSO, or NMP.

27. The method of claim 24, wherein the conditions sufficient to provide a compound of formula 3 comprise the presence of a base.

28. The method of claim 27, wherein the base is cesium carbonate, potassium carbonate, or potassium phosphate.
